(19) United States
(12) Patent Application Publication
(10) Pub. No.: US 2022/0347150 A1
(43) Pub. Date: Nov. 3, 2022

(54) TASIMELTEON USE IN TREATING SLEEP ABERRATIONS

(71) Applicant: Vanda Pharmaceuticals Inc., Washington, DC (US)

(72) Inventors: Mihael Polymeropoulos, Potomac, MD (US); Sandra SMIESZEK, Washington, DC (US)

(21) Appl. No.: 17/621,447

(22) PCT Filed: Jun. 29, 2020
(86) PCT No.: PCT/US2020/040081

§ 371 Date: Dec. 21, 2021

Related U.S. Application Data
(60) Provisional application No. 62/868,881, filed on Jun. 29, 2019.

(51) Int. Cl. A61K 31/343; A61P 25/20; A61K 31/55; A61K 33/00

(52) U.S. Cl. CPC A61K 31/343; A61P 25/20; A61K 31/55; A61K 33/00

(57) ABSTRACT

The invention relates generally to circadian rhythm disorders and, more particularly, to the treatment or prevention of circadian rhythm disorders based on an individual's HCN1 genotype. One aspect of the invention provides a method of treating an individual for delayed sleep time comprising: determining or having determined from a biological sample of the individual that the individual has a GG genotype at the rs12188518 single nucleotide polymorphism (SNP) locus; and administering to the individual once daily before a target bedtime a dose of tasimelteon effective to advance the sleep time of the individual. Other genotypes related with circadian rhythm disorders are CC at rs11248864 and AA at rs72762058.

### CROSS-REFERENCE TO RELATED APPLICATIONS

This application claims the benefit of co-pending U.S. Provisional Patent Application Ser. No. 62/868,881, filed 29 Jun. 2019, which is hereby incorporated in its entirety as though fully set forth.

### BACKGROUND OF THE INVENTION

The present invention provides new uses of tasimelteon in the treatment of sleep aberrations. In particular, the present invention relates to the use of tasimelteon in patients with certain identified genetic markers.

Circadian rhythms affect many aspects of human physiology, including a wide range of molecular and behavioral processes. Alterations in the timing or misalignment of circadian rhythms can result in various untoward effects.

The intrinsic period of the human circadian pacemaker averages greater than the 24-hour day, i.e., averages 24.18 hours. While the circadian pacemaker operates in healthy individuals to synchronize the individual's day-night sleep cycle to a 24-hour day with a normal daily timing for onset of the individual's sleep period, other “chronotypes” can produce aberrant day-night sleep cycles. Both “morning” and “evening” chronotypes are known sleep aberrations. For individuals exhibiting these chronotypes, there is a propensity for a daily early or delayed sleep timing, respectively, relative to the sleep timing that would normally be experienced by an individual with a synchronized circadian pacemaker (i.e. an individual with a natural chronotype or natural sleep time).

The evening chronotype (delayed onset of sleep time) has been associated with greater morbidity, including higher rates of metabolic dysfunction and cardiovascular disease. This chronotype is also associated with a higher incidence of type II diabetes and obesity, as compared to the morning chronotype (advanced onset of sleep time). The evening chronotype has also been associated with an increased likelihood of depression. Similar associations have been found among those engaged in shift work, where sleep is delayed with respect to an individual's natural chronotype or natural sleep timing.

Sleep aberrations also include having a circadian period (tau) that is shortened, i.e., less than 24 hours, such that sleep cycles are less than 24 hours apart.

Genetic variations underly a number of circadian rhythm and sleep disorders. Delayed Sleep Phase Disorder (DSPD), for example, has been associated with a CRY1 splicing variant. See Patke et al., Mutation of the Human Circadian Clock Gene CRY1 in Familial Delayed Sleep Phase Disorder, Cell 169(2):203-215.e13 (2017). Several large genome-wide association studies (GWASs) have found correlations between a number of chronotype-associated loci and mental health disorders. See Jones et al. Genome-wide association analyses of chronotype in 697,828 individuals provides insights into circadian rhythms, Nature Communications 10:343 (2019) and references cited therein.

Sleep aberrations may also include drug-induced delayed sleep time, where an individual exhibits a delayed sleep time similar to that observed in DSPD as a result of the effects of one or more active agents.

Hyperpolarization-activated cyclic nucleotide-gated (HCN) channels are integral membrane proteins that mediate rhythmic electrical activity of cardiac pacemaker cells and, in neurons, play important roles in setting resting membrane potentials, dendritic integration, neuronal pacemaking, and the establishment of action potential thresholds. Because of their role in generating rhythmic activity in heart and brain cells, HCN channels are often referred to as pacemaker channels. See Bender et al., Hyperpolarization activated cyclic-nucleotide gated (HCN) channels in developing neuronal networks, Prog. Neurobiol. 86(3):129-140 (2008).

In humans, four HCN channel proteins are known—HCN1, HCN2, HCN3, and HCN4. HCN1 is highly expressed in the brain and may modulate excitability in the brain. Specifically, the HCN1 protein is expressed in the frontal cortex and the retina and plays a critical role in shaping the autonomous activity of single neurons and the periodicity of network oscillations.

Tasimelteon (marketed under the trademark HETLIOZ®), and pharmaceutical compositions and uses thereof, have been described in the art. See U.S. Pat. No. 5,856,529 and the compound specifically described in claim 7 therein. Tasimelteon is approved for use as a human medicine for the treatment of Non-24-Hour Sleep-Wake Disorder (Non-24) and is available in a 20 mg unit pharmaceutical dosage form (capsules), indicated for use prior to bedtime at the same time every night. Pharmacologically, tasimelteon is an agonist of the MT1R and MT2R melatonin receptors in the suprachiasmatic nucleus (SCN), the region of the brain associated with the biological clock. Engagement of these receptors by melatonin is believed to regulate circadian rhythms, including the sleep/wake cycle. Consistent with its receptor binding profile, tasimelteon demonstrates potent chronobiotic activity in preclinical models of acute phase-shifting and chronic re-entrainment.

U.S. Pat. No. 5,856,529 further claims a genus of compounds of which tasimelteon is a member for use in treating sleep disorders, as well as circadian rhythm disorders, in patients by administering an effective amount of tasimelteon. The patent describes tasimelteon as a melatonin agonist and states that melatonin agonists would be useful for the further study of melatonin receptor interactions as well as in the treatment of conditions affected by melatonin activity. The patent lists depression, jet lag, work-shift syndrome, and sleep disorders, among other possible therapeutic uses. Elsewhere the patent discloses that compounds within the genus of compounds of which tasimelteon is a member are useful as melatonergic agents in the treatment of sleep disorders, seasonal depression, shifts in circadian cycles, melancholia, stress, appetite regulation, benign prostatic hyperplasia and related conditions.

In addition to tasimelteon's approved dosing of 20 mg per day prior to bedtime at the same time every day, WO2007/137244 reports the discovery that effective human doses for tasimelteon can range from 10 to 100 mg/day for contemplated uses in sleep disorders and circadian rhythm disorders, with a further description that the exact dosing may be dependent upon particle size of the tasimelteon and the body size of the patient being treated. The publication also describes a 20 mg oral unit dosage form for tasimelteon and a clinical trial using tasimelteon in 10 mg, 20 mg, 50 mg, and 100 mg daily doses.

In US Patent Application Publication No. 20090105333A1 (WO2007/137244), results are reported from the aforementioned clinical trial in which tasimelteon was studied in subjects with a 5-hour advance in their sleep-wake cycle, i.e., the type of sleep-wake cycle advance that might be experienced by a subject traveling by jet aircraft across the Atlantic Ocean from New York to London, including that treatment relative to placebo produced positive outcomes for shifting dim light melatonin onset and sleep efficacy.

### SUMMARY OF THE INVENTION

The present invention provides, in certain aspects, methods for treating individuals experiencing sleep aberrations. In particular, aspects of the present invention provide methods for use of tasimelteon for treating such individuals. The sleep aberrations addressed by the methods particularly relate to those individuals having certain single nucleotide polymorphisms (SNPs).

One aspect of the invention provides a method of treating an individual for delayed sleep time comprising: determining or having determined from a biological sample of the individual that the individual has a GG genotype at the rs12188518 single nucleotide polymorphism (SNP) locus; and administering to the individual once daily before a target bedtime a dose of tasimelteon effective to advance the sleep time of the individual.

Another aspect of the invention provides a method of treating an individual for delayed sleep time comprising: determining or having determined from a biological sample of the individual that the individual has a GG genotype at the rs12188518 single nucleotide polymorphism (SNP) locus; and administering to the individual once daily before a target bedtime a dose of tasimelteon effective to advance the sleep time of the individual.

Still another aspect of the invention provides a method of treating a patient for delayed sleep time, the improvement comprising: determining or having determined from a biological sample of the patient the patient's genotype at the rs12188518 single nucleotide polymorphism (SNP) locus, the rs11248864 SNP locus, or both; and in the case that the genotype of the patient is GG at the rs12188518 SNP locus, the genotype of the patient is CC at the rs11248864 SNP locus, or both, administering to the patient once daily a dose of tasimelteon effective to advance the patient's sleep time.

Still another aspect of the invention provides a method of delaying sleep time or delaying sleep phase in an individual, the method comprising: determining or having determined from a biological sample of the individual that the individual has an AA or AG genotype at the rs12188518 single nucleotide polymorphism (SNP) locus; and administering to the individual at least one hyperpolarization-activated cyclic nucleotide-gated (HCN) channel inhibitor.

Yet another aspect of the invention provides a method of delaying sleep time or delaying sleep phase in an individual, the method comprising: determining or having determined from a biological sample of the individual that the individual has a TT or TC genotype at the rs11248864 single nucleotide polymorphism (SNP) locus; and administering to the individual at least one hyperpolarization-activated cyclic nucleotide-gated (HCN) channel inhibitor.

Still yet another aspect of the invention provides a method of treating an individual determined to have a genotype predictive of an elongated circadian period (tau) based upon the individual having an AA genotype at the rs72762058 single nucleotide polymorphism (SNP) locus, the method comprising: administering to the individual once daily before a target bedtime a dose of tasimelteon effective to shorten the tau of the individual.

Yet another aspect of the invention provides a method of altering a circadian period (tau) in an individual, the method comprising: determining a genotype of the individual at the rs72762058 single nucleotide polymorphism (SNP) locus; and in the case that the genotype of the individual at the rs72762058 SNP locus is AA, administering to the individual a quantity of tasimelteon effective to decrease the tau of the individual.

Another aspect of the invention provides a method of treating a patient having a delayed sleep phase who is being treated with a hyperpolarization-activated cyclic nucleotide-gated (HCN) channel inhibitor, the method comprising: administering to the patient once daily a dose of tasimelteon effective to advance the patient's sleep time.

Still another aspect of the invention provides, in a method of treating an individual for a circadian rhythm disorder, the improvement comprising: determining a genotype of the individual at the rs72762058 single nucleotide polymorphism (SNP) locus; and in the case that the genotype of the individual at the rs72762058 SNP locus is AA, administering to the individual a quantity of tasimelteon effective to shorten a circadian period (tau) of the individual.

And yet another aspect of the invention provides, in a method of entraining a light perception impaired patient suffering from Non-24-Hour Sleep-Wake Disorder to a 24-hour sleep-wake cycle in which the patient awakens at or near a target wake time following a daily sleep period of approximately 7 to 9 hours, wherein the patient is being treated with a hyperpolarization-activated cyclic nucleotide-gated (HCN) channel inhibitor, the improvement comprising: discontinuing treatment of the patient with the HCN channel inhibitor; and, then orally treating the patient with 20 mg of tasimelteon once daily before a target bedtime for a duration of treatment effective to entrain said patient to the 24-hour sleep-wake cycle, thereby avoiding the use of tasimelteon in combination with an HCN channel inhibitor.

The individuals being treated in accordance with the methods herein are human beings. In particular, the individuals being treated are those with a particular genotype at one or more of the rs12188518; rs72762058, or rs 11248864 SNP loci. These SNPs, identified by their “rs” SNP locus designations, are found on human chromosome 5, i.e., chr5:46086964 (GRCh38.p12) (A>G; A>T) and chr5:45467324 (GRCh38.p12) (G>A), or chromosome 16 (i.e., chr16:1303514 (GRCh38.p12) (T>C; T>G). See, e.g., https://www.ncbi.nlm.nih.gov/snp/rs12188518, https://www.ncbi.nlm.nih.gov/snp/rs72762058, and https://www.ncbi.nlm.nih.gov/snp/rs11248864, respectively.

One skilled in the art will understand that the “rs” designators employed herein refer to “reference SNP ID” numbers, an identification system employed by NCBI to refer to a group or cluster of SNPs that map to an identical location. Other identification systems may be employed to refer to these chromosomal locations, as will be appreciated by one skilled in the art.

As used herein the term “sleep aberration” can include, for example, an abnormal pattern of sleep in an individual, which may take the form of a pattern of abnormally delayed onset of sleep or sleepiness (i.e., “sleep time”), an abnormally early onset of sleep time, an abnormally long or abnormally short daily sleep period (i.e., substantially less than or substantially greater than a normal daily period of 7-9 hours in duration that an individual would set aside daily for sleep), or abnormal periods of prolonged daytime sleep or sleepiness as part of the individual's day-night sleep cycle.

One common form of sleep aberration results when an individual has a pattern of delayed sleep time relative to the sleep time that would normally be appropriate for a day-night sleep cycle for the individual. As noted above, “sleep aberration” may also include a shortened (i.e. less than 24-hour) tau or drug-induced delayed sleep time.

The determination of an individual's SNP status at a particular SNP locus is undertaken by methods known in the art for determining single nucleotide polymorphisms. In this regard, standard genetic testing is done using known techniques for analyzing a biological sample from an individual.

The treatment regimen described herein of administering tasimelteon is undertaken by administration before bedtime, using treatment regimens known in the art for the treatment of individuals with non-24. In this regard, the administration can be undertaken up to about 2 hours before the individual's target bedtime. Most typically the administration is undertaken about 0.5 to 1.5 hours before the target bedtime, e.g., about 1 hour before the target bedtime.

The administration of tasimelteon orally is preferred. Typically, a solid oral dosage form is used, e.g., a single capsule containing the daily dose of tasimelteon to be administered, although other routes of administration and dosage forms providing an equivalent effect can be used.

The amount of tasimelteon can vary based upon an individual's clinical response, although daily doses of 10 mg to 100 mg are typically effective under the treatment regimens herein. The preferred doses for treatment of adults are typically 20 mg to 50 mg daily, with 20 mg daily representing a typical effective dose.

When the tasimelteon is administered using an oral dosage form, any of the typical methods for administration can be used. Suitable dosage forms include liquid oral dosage forms and solid oral dosage forms, such as conventional compressed tablets and capsules.

### DETAILED DESCRIPTION

The present invention can be further understood through the following examples.

### Example 1

Chronotype Prediction

A first whole genome sequencing study is conducted using 316 samples collected as part of a clinical study directed to the effects of abrupt circadian advance, as may be experienced during eastward jet travel, and the ability of tasimelteon to mitigate or eliminate those effects. Participants in that study are healthy, sighted individuals not known to be suffering from a circadian rhythm disorder or sleep disorder.

More than 400 single nucleotide polymorphisms (SNPs) in linkage disequilibrium are found. Among the SNPs that are identified in this study, rs121888518, located within the HCN1 gene on chromosome 5, is found to be predictive of evening chronotype when compared to individuals' Morningness-Eveningness Questionnaire (MEQ) scores. Specifically, individuals with a GG genotype at the rs121888518 SNP locus are significantly more likely to be classified as having an evening chronotype based on MEQ score. This association is persistent in a binary analysis (logistic regression) and when using alternative morningness-eveningness questionnaires. Expression quantitative trait locus (eQTL) analysis indicates a significant result (1.6·10⁹) for the rs121888518 SNP

These results are consistent with a mechanism of action by which individuals exhibit an evening chronotype.

The HCN1 channel is responsible for feedback on the rods, regulating the dynamic range of light reactivity under dim or intermediate light conditions. Proper cone vision under mesopic conditions requires rapid adaptational feedback modulation of rod output via the HCN1 channels. When these channels are absent or inhibited, sustained rod responses following bright light exposure saturate the retinal network, resulting in a loss of downstream cone signaling.

The improper functioning of this feedback system in an individual may result in rod saturation, even in dim light. This may result in a misperception of light conditions and a consequent circadian delay, experienced as a sleep aberration, which may include delayed sleep time.

Thus, resetting the circadian period of an individual with a genetic predisposition to having an evening chronotype (e.g., GG genotype at the rs121888518 SNP locus) provides an avenue to eliminate or ameliorate the consequences of such a sleep aberration. Administering to such an individual a dose of tasimelteon before a target bedtime operates to advance the sleep time in the individual, thereby treating the sleep aberration.

Another SNP, rs11248864, located on chromosome 16, similarly exhibits a significant correlation (p<10⁻⁸) with chronotype. Specifically, individuals having a CC genotype at the rs11248864 SNP locus are significantly more likely to be classified as having an evening chronotype based on MEQ score (eQTL=1.3·10⁸).

The rs11248864 SNP is located within the Ubiquitin Conjugating Enzyme E2 I (UBE2I) gene. The UBE2I enzyme directly interacts with transcriptional repressor BHLHE40 and is potentially of direct relevance to the input and output of the circadian clock. The UBE2I region is also a significant eQTL for brain-specific angiogenesis inhibitor (BAIAP3), which in turn likely plays a role in hypothalamic neuronal firing by modulating gamma-aminobutyric acid (GABA)ergic inhibitory neurotransmission and is highly expressed in the pituitary.

As noted above, resetting the circadian period of an individual with a genetic predisposition to having an evening chronotype provides a method to eliminate or ameliorate the consequences of such a sleep aberration. Thus, administering to such an individual (e.g., one having a CC genotype at the rs11248864 SNP locus) a dose of tasimelteon before a target bedtime operates to advance the sleep time in the individual, thereby treating the sleep aberration.

This mechanism of action also suggests a method by which to delay sleep time in an individual. For example, an individual not predicted to have an evening chronotype (e.g., an individual having an AA or AG genotype at the rs12188518 SNP locus or a TT or TC genotype at the rs11248864 SNP locus) may be administered an HCN channel inhibitor in an amount effective to induce in such an individual a saturation of the rods under dim or intermediate light conditions, resulting in a delayed sleep time. Suitable HCN inhibitors include, for example, ivabradine, cilobradine, zatebradine, or cesium.

### Example 2

HCN1 and Prolonged Tau

A second whole genome sequencing study is conducted of 174 totally blind individuals with Non-24-Hour Sleep-Wake Disorder (Non-24). Non-24 is a circadian rhythm disorder in which the master body clock runs either slightly shorter or, more commonly, slightly longer than 24 hours.

Within this group, an association is found between HCN1 variants and circadian period length (tau) as calculated from the measurement of urinary 6-sulphatoxymelatonin (aMT6s) rhythms, aMT6s being the major metabolite of melatonin. Specifically, the minor allele of the SNP rs72762058, representing a G-to-A mutation at position 45467426, shows a significant association with longer tau. Individuals with this mutation have a mean tau of 24.71 hours, 12 minutes longer than those not having the minor allele.

Thus, shortening the tau of an individual shown to have a genetic predisposition to having a long tau provides a method for treating circadian rhythm and sleep aberrations associated with a long tau. Administering to such an individual an effective amount of tasimelteon operates to shorten the individual's tau, resulting in elimination or amelioration of the circadian rhythm or sleep aberration.

The description of the present disclosure is presented for purposes of illustration and description but is not intended to be exhaustive or limited to the disclosure in the form disclosed. Many modifications and variations will be apparent to those of ordinary skill in the art without departing from the scope and spirit of the disclosure.

We claim:

1. A method of treating an individual for delayed sleep time comprising:
determining or having determined from a biological sample of the individual that the individual has a GG genotype at the rs12188518 single nucleotide polymorphism (SNP) locus; and
administering to the individual once daily before a target bedtime a dose of tasimelteon effective to advance the sleep time of the individual.

2. The method of claim 1, wherein the administration is oral.

3. The method of claim 1, wherein the administration comprises an oral dose 0.5 hour to 2 hours before the individual's target bedtime.

4. The method of claim 1, wherein the dose is between about 10 mg and about 100 mg.

5. The method of claim 3, wherein the dose is between about 20 mg and about 50 mg.

6. The method of claim 4, wherein the dose is 20 mg.

7. A method of treating an individual for delayed sleep time comprising:
determining or having determined from a biological sample of the individual that the individual has a CC genotype at the rs11248864 single nucleotide polymorphism (SNP) locus; and
administering to the individual once daily before a target bedtime a dose of tasimelteon effective to advance the sleep time of the individual.

8. The method of claim 7, wherein the administration is oral.

9. The method of claim 7, wherein the administration comprises an oral dose 0.5 hour to 2 hours before the target bedtime.

10. The method of claim 7, wherein the dose is between about 10 mg and about 100 mg.

11. The method of claim 10, wherein the dose is between about 20 mg and about 50 mg.

12. The method of claim 11, wherein the dose is 20 mg.

13. In a method of treating a patient for delayed sleep time, the improvement comprising:
determining or having determined from a biological sample of the patient the patient's genotype at the rs12188518 single nucleotide polymorphism (SNP) locus, the rs11248864 SNP locus, or both; and
in the case that the genotype of the patient is GG at the rs12188518 SNP locus, the genotype of the patient is CC at the rs11248864 SNP locus, or both, administering to the patient once daily a dose of tasimelteon effective to advance the patient's sleep time.

14. The improvement of claim 13, wherein the administration comprises an oral dose 0.5 hour to 2 hours before a target bedtime.

15. The improvement of claim 14, wherein the dose is about 10 mg to about 100 mg.

16. The improvement of claim 15, wherein the dose is about 20 mg to about 50 mg.

17. The improvement of claim 16, wherein the dose is about 20 mg.

18. The improvement of claim 13, wherein the dose administered to the patient is greater than would be administered to an individual to advance sleep time not having a genotype indicative of a predisposition to having a delayed sleep time.

19. A method of delaying sleep time or delaying sleep phase in an individual, the method comprising:
determining or having determined from a biological sample of the individual that the individual has an AA or AG genotype at the rs12188518 single nucleotide polymorphism (SNP) locus; and
administering to the individual at least one hyperpolarization-activated cyclic nucleotide-gated (HCN) channel inhibitor.

20. The method of claim 19, wherein the at least one HCN channel inhibitor is selected from a group consisting of: ivabradine, cilobradine, zatebradine, and cesium.

21. A method of delaying sleep time or delaying sleep phase in an individual, the method comprising:
determining or having determined from a biological sample of the individual that the individual has a TT or TC genotype at the rs11248864 single nucleotide polymorphism (SNP) locus; and
administering to the individual at least one hyperpolarization-activated cyclic nucleotide-gated (HCN) channel inhibitor.

22. The method of claim 21, wherein the at least one HCN channel inhibitor is selected from a group consisting of: ivabradine, cilobradine, zatebradine, ZD7299, and cesium.

23. A method of treating an individual determined to have a genotype predictive of an elongated circadian period (tau) based upon the individual having an AA genotype at the rs72762058 single nucleotide polymorphism (SNP) locus, the method comprising:
administering to the individual once daily before a target bedtime a dose of tasimelteon effective to shorten the tau of the individual.

24. The method of claim 23, wherein the administration comprises an oral dose 0.5 hour to 2 hours before the target bedtime.

25. The method of claim 23, wherein the dose is about 10 mg to about 100 mg.

26. The method of claim 25, wherein the dose is about 20 mg to about 50 mg.

27. The method of claim 26, wherein the dose is about 20 mg.

28. A method of altering a circadian period (tau) in an individual, the method comprising:
determining a genotype of the individual at the rs72762058 single nucleotide polymorphism (SNP) locus; and
in the case that the genotype of the individual at the rs72762058 SNP locus is AA, administering to the individual a quantity of tasimelteon effective to decrease the tau of the individual.

29. The method of claim 28, wherein the administration is oral.

30. The method of claim 28, wherein the administration comprises an oral dose 0.5 hour to 2 hours before the individual's target bedtime.

31. The method of claim 28, wherein the dose is between about 10 mg and about 100 mg.

32. The method of claim 31, wherein the dose is between about 20 mg and about 50 mg.

33. The method of claim 32, wherein the dose is 20 mg.

34. A method of treating a patient having a delayed sleep phase who is being treated with a hyperpolarization-activated cyclic nucleotide-gated (HCN) channel inhibitor, the method comprising:
administering to the patient once daily a dose of tasimelteon effective to advance the patient's sleep time.

35. The method of claim 34, wherein the HCN channel inhibitor is selected from a group consisting of: ivabradine, cilobradine, zatebradine, ZD7299, and cesium.

36. The method of claim 34, wherein the administration is oral.

37. The method of claim 34, wherein the administration comprises an oral dose 0.5 hour to 2 hours before the individual's target bedtime.

38. The method of claim 34, wherein the dose is between about 10 mg and about 100 mg.

39. The method of claim 38, wherein the dose is between about 20 mg and about 50 mg.

40. The method of claim 39, wherein the dose is 20 mg.

41. In a method of treating an individual for a circadian rhythm disorder, the improvement comprising:
determining a genotype of the individual at the rs72762058 single nucleotide polymorphism (SNP) locus; and
in the case that the genotype of the individual at the rs72762058 SNP locus is AA, administering to the individual a quantity of tasimelteon effective to shorten a circadian period (tau) of the individual.

42. The method of claim 41, wherein the administration is oral.

43. The method of claim 41, wherein the administration comprises an oral dose 0.5 hour to 2 hours before the individual's target bedtime.

44. The method of claim 41, wherein the dose is between about 10 mg and about 100 mg.

45. The method of claim 44, wherein the dose is between about 20 mg and about 50 mg.

46. The method of claim 45, wherein the dose is 20 mg.
